(19) United States
(12) Patent Application Publication
(10) Pub. No.: US 2016/0000689 A1
(43) Pub. Date: Jan. 7, 2016

(54) COSMETIC COMPOSITION COMPRISING A VINYLFORMAMIDE/VINYLFORMAMINE COPOLYMER, A NON-IONIC FIXING POLYMER AND A CATIONIC SURFACTANT

(71) Applicant: L'OREAL, Paris (FR)

(72) Inventors: Dorothee PASQUET, Bois Colombes (FR); Christine ANNOTEL, Paris (FR); Cecile BEBOT, Asnieres (FR)

(21) Appl. No.: 14/767,925

(22) PCT Filed: Feb. 12, 2014
(86) PCT No.: PCT/EP2014/052740

§ 371 Date: Aug. 14, 2015

Related U.S. Application Data
(60) Provisional application No. 61/773,196, filed on Mar. 6, 2013.

(30) Foreign Application Priority Data
Feb. 15, 2013 (FR) 1351318

(51) Int. Cl. A61K 8/81; A61Q 5/06; A61K 8/04

(52) U.S. Cl. CPC A61K 8/8158; A61K 8/046; A61Q 5/06

(57) ABSTRACT

The present invention relates to a cosmetic composition comprising: one or more vinylformamide/vinylformamine copolymers comprising: from 10 to 95 mol % of units of formula A and from 90 to 5 mol % of units of formula B, one or more non-ionic fixing polymers, and one or more cationic surfactants. It also relates to the use of this composition for shaping and/or fixing the hairstyle, to a cosmetic treatment process for the hair using same and also to a particular cosmetic set.

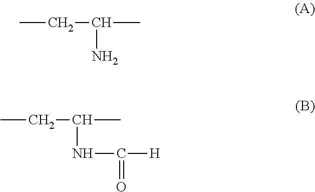

The present invention relates to a cosmetic composition comprising one or more vinylformamide/vinylformamine copolymers, one or more non-ionic fixing polymers and one or more cationic surfactants.

The invention also relates to a cosmetic treatment process for the hair, in particular a process for fixing and/or shaping the hair, using the abovementioned composition.

Finally, the invention relates to a use of this composition for cosmetic hair treatment, and in particular for styling hair, i.e. shaping and/or fixing the hairstyle.

Styling products are normally used to construct and structure the hairstyle and to give it shape retention. They are usually in the form of lotions, gels, foams, creams, sprays, etc. The corresponding compositions generally comprise one or more film-forming polymers or “fixing polymers”. These polymers allow the formation of a coating film on the hair, thus providing form retention of the hairstyle.

However, the fixing-polymer films thus formed have the drawback of being relatively friable, thereby limiting the shape retention of the hairstyle, and causing the formation of unaesthetic residues on the hair.

Thus, conventional styling products result in fixing of the hairstyle and styling effects which gradually fade over time. In particular, when the product is applied in the morning, the styling effects fade over the course of the day. The following day, the styling effects are weak, or even non-existent.

To remedy this problem, it is known practice to incorporate, into styling products, polymers which have a very high fixing capacity, and/or to increase the concentration of fixing polymer. However, the use of such extremely fixing products leads to a certain number of drawbacks. In particular, these products result in the hair having a dry and rough feel and they are difficult to remove with shampoo.

Patent application FR 2 926 989 describes compositions comprising vinylformamide/vinylformamine copolymers and anionic or non-ionic polymers, for in particular producing styling gels.

Moreover, when foams are used, they have more or less firm textures with limited hold in the hand.

There is therefore a need for hair compositions which make it possible to obtain long-lasting fixing of the hairstyle, with styling effects that last throughout the day or even for several days, while at the same time being easy to remove with shampoo and providing a pleasant cosmetic feel, and in particular a smooth feel.

There is also a need to obtain foams with an improved texture.

The applicant has now discovered, surprisingly, that the combination of a vinylformamide/vinylformamine copolymer with a non-ionic fixing polymer and a cationic surfactant in a cosmetic composition makes it possible to obtain a cosmetic hair composition which provides improved styling properties. In particular, such a combination makes it possible to obtain styling products which provide long-lasting fixing of the hairstyle, while at the same time being easy to remove and giving the hair a pleasant cosmetic feel. It also makes it possible to obtain a composition in the form of a foam with an expanded texture which is easy to hold.

A subject of the present invention is thus a cosmetic composition comprising:

one or more vinylformamide/vinylformamine copolymers comprising:

from 10 to 95 mol % of units of the following formula A:

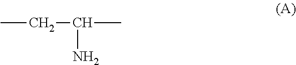

and from 90 to 5 mol % of units of the following formula B:

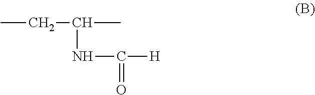

one or more non-ionic fixing polymers; and

one or more cationic surfactants.

Advantageously, this composition is non-washing, i.e. it contains less than 5% by weight in total of anionic surfactants and of non-ionic surfactants.

The compositions according to the invention make it possible to obtain styling compositions which provide long-lasting fixing and make it possible in particular to obtain expanded foams which have good styling and cosmetic properties.

Other subjects, characteristics, aspects and advantages of the invention will become even more clearly apparent on reading the description and examples that follow.

According to the invention, the cosmetic composition comprises one or more vinylformamide/vinylformamine copolymers, one or more non-ionic fixing polymers and one or more cationic surfactants.

### Vinylformamide/Vinylformamine Copolymers

The vinylformamide/vinylformamine copolymer(s) which can be used in the compositions according to the invention preferably comprise(s) from 10 to 60 mol % of units of formula A and more particularly from 20 to 40 mol %.

The vinylformamide/vinylformamine copolymer(s) according to the invention preferably comprise(s) from 30 to 90 mol % of units of formula B and more particularly from 60 to 80 mol %.

The copolymers according to the invention can be obtained, for example, by partial hydrolysis of polyvinylformamide. This hydrolysis may be performed in an acidic or basic medium.

The vinylformamide/vinylformamine copolymer(s) according to the invention can optionally comprise one or more additional monomer units. In this case, the latter preferably represent less than 20 mol % of the copolymer.

According to one preferred embodiment, the vinylformamide/vinylformamine copolymer(s) according to the invention consist(s) solely of units of formula A and of units of formula B.

The weight-average molecular weight of said copolymer, measured by light diffraction, can vary from 10 000 to 30 000 000 g/mol, preferably from 40 000 to 1 000 000 g/mol and more particularly from 100 000 to 500 000 g/mol.

The cationic charge density of said copolymer can vary from 2 meq/g to 20 meq/g, preferably from 2.5 to 15 meq/g and more particularly from 3.5 to 10 meq/g.

By way of example of vinylformamide/vinylformamine copolymers that can be used in the compositions according to the invention, mention will be made, inter alia, of the products sold under the name Luviquat 9030 by the company BASF, and the products provided under the name Lupamin 9010, Lupamin 5095 and Lupamin 1595 by the company BASF.

The vinylformamide/vinylformamine copolymer(s) is (are) present in the compositions according to the invention in proportions preferably ranging from 0.01% to 20% by weight, more preferentially from 0.1% to 10% by weight and more particularly from 0.1% to 5% by weight, relative to the total weight of the composition.

### Non-Ionic Fixing Polymers

As indicated previously, the cosmetic composition according to the invention comprises one or more non-ionic fixing polymers.

The term “fixing polymer” is intended to mean any polymer capable of conferring a shape on a head of hair or of maintaining a head of hair in a given shape.

The non-ionic fixing polymers that can be used according to the present invention are chosen, for example, from:

polyalkyloxazolines;

vinyl acetate homopolymers;

vinyl acetate copolymers, for instance copolymers of vinyl acetate and acrylic ester, copolymers of vinyl acetate and ethylene, or copolymers of vinyl acetate and maleic ester, for example dibutyl maleate;

homopolymers and copolymers of acrylic esters, for instance copolymers of alkyl acrylates and of alkyl methacrylates, such as the products sold by the company Röhm & Haas under the names Primal® AC-261 K and Eudragit® NE 30 D, by the company BASF under the name 8845, or by the company Hoechst under the name Appretan® N9212;

copolymers of acrylonitrile and a non-ionic monomer chosen, for example, from butadiene and alkyl(meth)acrylates, such as the products provided under the name CJ 0601 B by the company Röhm & Haas;

styrene homopolymers;

styrene copolymers, for instance copolymers of styrene and of alkyl(meth)acrylate, such as the products Mowilith® LDM 6911, Mowilith® DM 611 and Mowilith® LDM 6070 sold by the company Hoechst, and the products Rhodopas® SD 215 and Rhodopas® DS 910 sold by the company Rhône-Poulenc, copolymers of styrene, of alkyl methacrylate and of alkyl acrylate, copolymers of styrene and of butadiene, or copolymers of styrene, of butadiene and of vinylpyridine;

polyamides;

vinyllactam homopolymers, such as vinylpyrrolidone homopolymers, such as the polyvinylcaprolactam sold under the name Luviskol® PLUS by the company BASF, or the polyvinylpyrrolidone sold under the name PVP K30 L by the company ISP;

vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF, and poly(vinylpyrrolidone/vinyl acetate/vinyl propionate) terpolymers, for instance the product sold under the name Luviskol® VAP 343 by the company BASF; and

poly(vinyl alcohols).

The alkyl groups of the non-ionic polymers mentioned above preferably have from 1 to 6 carbon atoms.

The non-ionic fixing polymer(s) used according to the invention is (are) preferably chosen from vinyllactam homopolymers, such as vinylpyrrolidone homopolymers, polyvinylcaprolactam, and vinyllactam copolymers, such as a poly(vinylpyrrolidone/vinyllactam) copolymer, poly(vinylpyrrolidone/vinyl acetate) copolymers, and poly(vinylpyrrolidone/vinyl acetate/vinyl propionate) terpolymers.

More preferentially, the non-ionic fixing polymer(s) used according to the invention is (are) chosen from vinyllactam homopolymers and copolymers, such as vinylpyrrolidone homopolymers and poly(vinylpyrrolidone/vinyl acetate) copolymers.

The non-ionic fixing polymer(s) is (are) present in an amount preferably ranging from 0.1% to 20% by weight, even better still from 0.2% to 10% by weight and even more preferentially from 0.5% to 5% by weight relative to the total weight of the composition.

### Cationic Surfactants

As indicated previously, the composition according to the invention comprises one or more cationic surfactants as defined hereinafter.

Generally, a cationic surfactant may bear one or more permanent positive charges or may comprise one or more functional groups that can form cations in the composition according to the invention. The cationic surfactants of the invention are preferably those bearing one or more permanent positive charges, i.e. one or more quaternized nitrogen atoms.

The cationic surfactants that may be used in the composition according to the invention comprise quaternary ammonium salts, and mixtures thereof.

Examples of quaternary ammonium salts that may especially be mentioned include:

(A) those corresponding to formula (I) below:

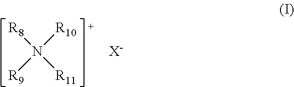

in which:

the radicals R₈ to R₁₁, which may be identical or different, represent a saturated or unsaturated, linear or branched aliphatic hydrocarbon-based radical comprising from 1 to 30 carbon atoms, or an aromatic radical such as aryl or alkylaryl, at least one of the radicals R₈ to R₁₁ comprising from 8 to 30 carbon atoms and preferably from 12 to 24 carbon atoms; it being possible for the aliphatic radicals to comprise heteroatoms such as oxygen, nitrogen, sulfur or halogens.

The aliphatic radicals are chosen, for example, from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl and C₁-C₃₀ hydroxyalkyl radicals;

X⁻ is an anion chosen from the group of halides, phosphates, acetates, lactates, (C₁-C₄)alkyl sulfates and (C₁-C₄)alkylsulfonates and (C₁-C₄)alkylarylsulfonates.

Among the quaternary ammonium salts of formula (I), preference is given, on the one hand, to the salts, in particular the chlorides, of dialkyldimethylammonium, of alkyltrimethylammonium or of alkenyldimethylhydroxyethylammonium in which the alkyl or alkenyl radical comprises approximately from 12 to 22 carbon atoms, in particular the salts, especially the chlorides, of behenyltrimethylammonium, of distearyldimethylammonium, of cetyltrimethylammonium, of dicetyldimethylammonium, or of oleylhydroxyethyldimethylammonium, the salts, in particular the chlorides, of aralkyldimethylalkylammonium, such as benzyldimethylstearylammonium chloride; and on the other hand, the salts, in particular the chlorides, of alkylamidoalkyltrimethylammonium, in particular palmitylamidopropyltrimethylammonium chloride;

(B) quaternary ammonium salts of imidazoline, for instance those of formula (II) below:

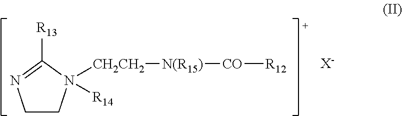

in which:

R₁₂ represents an alkenyl or alkyl radical comprising from 8 to 30 carbon atoms, for example tallow fatty acid derivatives,

R₁₃ represents a hydrogen atom, a C₁-C₄ alkyl radical or an alkenyl or alkyl radical comprising from 8 to 30 carbon atoms,

R₁₄ represents a C₁-C₄ alkyl radical,

R₁₅ represents a hydrogen atom or a C₁-C₄ alkyl radical,

X⁻ is an anion chosen from the group of halides, phosphates, acetates, lactates, (C₁-C₄)alkyl sulfates, (C₁-C₄)alkylsulfonates and (C₁-C₄)alkylarylsulfonates.

Preferably, R₁₂ and R₁₃ denote a mixture of alkenyl or alkyl radicals comprising from 12 to 21 carbon atoms, for example fatty acid derivatives of tallow, R₁₄ denotes a methyl radical and R₁₅ denotes a hydrogen atom. Such a product is sold, for example, under the name Varisoft W 575 PG N by the company Evonik Goldschmidt;

(C) diquaternary or triquaternary ammonium salts, in particular of formula (III) below:

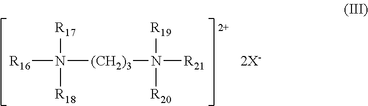

in which:

R₁₆ denotes an alkyl radical comprising approximately from 16 to 30 carbon atoms, which is optionally hydroxylated and/or interrupted with one or more oxygen atoms;

R₁₇ is chosen from hydrogen and an alkyl radical comprising from 1 to 4 carbon atoms or a group —(CH₂)₃—N+(R₁₆ₐ)(R₁₇ₐ)(R₁₈ₐ),

R₁₆ₐ, R₁₇ₐ, R₁₈ₐ, R₁₈, R₁₉, R₂₀ and R₂₁, which may be identical or different, are chosen from hydrogen and an alkyl radical comprising from 1 to 4 carbon atoms, and

X⁻ is an anion chosen from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P, sold by the company Innospec Active Chemicals (Quaternium 89), and Condicare CT sold by the company Innospec Active Chemicals (Quaternium 75);

(D) quaternary ammonium salts containing one or more ester functions, such as those of formula (IV) below:

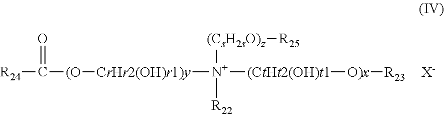

in which:

R₂₂ is chosen from C₁-C₆ alkyl radicals and C₁-C₆ hydroxyalkyl or dihydroxyalkyl radicals;

R₂₃ is chosen from:

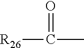

the radical

linear or branched, saturated or unsaturated C₁-C₂₂ hydrocarbon-based radicals R₂₇,

a hydrogen atom,

R₂₅ is chosen from:

the radical

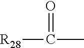

saturated or unsaturated, linear or branched C₁-C₆ hydrocarbon-based radicals R₂₉,

a hydrogen atom,

R₂₄, R₂₆ and R₂₈, which may be identical or different, are chosen from linear or branched, saturated or unsaturated C₇-C₂₁ hydrocarbon-based radicals;

r, s and t, which may be identical or different, are integers ranging from 2 to 6;

r1 and t1, which may be identical or different, are equal to 0 or 1;

r2+r1=2r and t1+t2=2t

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₂₃ denotes R₂₇ and that when z is 0, then R₂₅ denotes R₂₉.

The alkyl radicals R₂₂ may be linear or branched, and more particularly linear.

R₂₂ preferably denotes a methyl, ethyl, hydroxyethyl or dihydroxypropyl radical, and more particularly a methyl or ethyl radical.

Advantageously, the sum x+y+z is from 1 to 10.

When R₂₃ is a hydrocarbon-based radical R₂₇, it may be long and may contain from 12 to 22 carbon atoms, or may be short and may contain from 1 to 3 carbon atoms.

When R₂₅ is a hydrocarbon-based radical R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide, preferably chloride, bromide or iodide, a (C₁-C₄)alkyl sulfate or a (C₁-C₄)alkyl- or (C₁-C₄)alkylaryl-sulfonate. However, use may be made of methanesulfonate, phosphate, nitrate, tosylate, an anion derived from an organic acid, such as acetate or lactate, or any other anion compatible with ammonium bearing an ester function. The anion X⁻ is more particularly chloride, methyl sulfate or ethyl sulfate.

Use is made more particularly, in the composition according to the invention, of the ammonium salts of formula (IV) in which:

R₂₂ denotes a methyl or ethyl radical,

x and y are equal to 1;

z is equal to 0 or 1;

r, s and t are equal to 2;

R₂₃ is chosen from methyl, ethyl or C₁₄-C₂₂ hydrocarbon-based radicals and a hydrogen atom, or

the radical

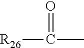

R₂₅ is chosen from a hydrogen atom, or

the radical

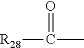

R₂₄, R₂₆ and R₂₈, which may be identical or different, are chosen from linear or branched, saturated or unsaturated C₁₃-C₁₇ hydrocarbon-based radicals, and preferably from linear or branched, saturated or unsaturated C₁₃-C₁₇ alkyl and alkenyl radicals.

Advantageously, the hydrocarbon-based radicals are linear.

Among the compounds of formula (IV), examples that may be mentioned include salts, especially the chloride or methyl sulfate, of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl radicals preferably contain 14 to 18 carbon atoms and are obtained more particularly from a plant oil such as palm oil or sunflower oil. When the compound contains several acyl radicals, these radicals may be identical or different.

Mention may be made more particularly of salts, and especially distearoylethylhydroxyethylmethylammonium, dipalmitoylethylhydroxyethylammonium or distearoylethylhydroxyethylammonium methosulfate.

These products are obtained, for example, by direct esterification of triethanolamine, triisopropanolamine, an alkyldiethanolamine or an alkyldiisopropanolamine, which are optionally oxyalkylenated, with fatty acids or with mixtures of fatty acids of plant or animal origin, or by transesterification of the methyl esters thereof. This esterification is followed by a quaternization by means of an alkylating agent such as an alkyl halide, preferably a methyl or ethyl halide, a dialkyl sulfate, preferably a methyl or ethyl sulfate, methyl methanesulfonate, methyl para-toluenesulfonate, glycol chlorohydrin or glycerol chlorohydrin.

The composition according to the invention may contain, for example, a mixture of quaternary ammonium monoester, diester and triester salts with a weight majority of diester salts.

It is also possible to use the ammonium salts containing at least one ester function that are described in U.S. Pat. No. 4,874,554 and U.S. Pat. No. 4,137,180. Use may be made of behenoylhydroxypropyltrimethylammonium chloride, sold by Kao under the name Quartamin BTC 131.

Preferably, the ammonium salts containing at least one ester function contain two ester functions.

Among the cationic surfactants that may be used in the composition according to the invention, the ones more particularly preferred are cetyltrimethylammonium, behenyltrimethylammonium, dipalmitoylethylhydroxyethylmethylammonium, oleylhydroxyethyldimethylammonium or methylalkylalkylamidoethylimidazolium salts, and mixtures thereof, and more particularly behenyltrimethylammonium chloride, sold for example by the company Clariant under the trade name Genamin KDMP or Genamin BTLF or by the company Evonik Goldschmidt under the name Varisoft BT 85, cetyltrimethylammonium chloride sold, for example, under the trade name Dehyquart A OR by the company Cognis or Quartamin 60 W25 by the company Kao or alternatively Genamin CTAC 25 by the company Clariant, and also methylalkylalkylamidoethylimidazolium methosulfate, such as the product sold under the name Varisoft W 575 PG N by the company Evonik Goldschmidt and oleylhydroxyethyldimethylammonium chloride sold under the name Chimexane CL by the company Chimex, and mixtures thereof.

Preferably, the cationic surfactants of the invention are chosen from the compounds of formula (I), (II) or (IV).

The composition according to the invention preferably comprises the cationic surfactant(s) in an amount ranging from 0.01% to 20% by weight, in particular from 0.1% to 10% by weight and better still from 0.15% to 5% by weight, relative to the total weight of the composition.

The composition according to the invention preferably comprises a cationic surfactant(s)/non-ionic fixing polymer(s) weight ratio ranging from 0.01 to 2 and even better still from 0.05 to 1.

The composition according to the invention preferably comprises an aqueous phase. The water content preferably ranges from 10% to 98%, preferably from 20% to 96%, better still from 50% to 96% by weight and even better still from 70% to 96% by weight relative to the total weight of the composition.

The composition may also comprise one or more organic solvents, such as C₁-C₄ lower alcohols, such as ethanol, isopropanol, tert-butanol or n-butanol; polyols such as propylene glycol; polyol ethers; C₅-C₁₀ alkanes; C₃-C₄ ketones such as acetone and methyl ethyl ketone; C₁-C₄ alkyl acetates such as methyl acetate, ethyl acetate and butyl acetate; dimethoxyethane, diethoxyethane; and mixtures thereof.

The composition of the invention may also comprise at least one customary cosmetic ingredient, in particular chosen from anionic, amphoteric and non-ionic surfactants, oils; solid fatty substances and in particular C₈-C₄₀ esters, C₈-C₄₀ acids; C₈-C₄₀ alcohols; sunscreens; moisturizing agents; antidandruff agents; antioxidants; chelating agents; pearlescent agents and opacifiers; plasticizers or coalescent agents; fillers, in particular inorganic fillers; glitter flakes; silicones, in particular silicone gums, alkoxylated or non-alkoxylated silicones; polymeric or non-polymeric thickeners or gelling agents; emulsifiers; polymers, other than those previously mentioned, in particular conditioning polymers, anionic, cationic or amphoteric fixing polymers; fragrances; preservatives; basifying agents such as sodium hydroxide, or acidifying agents; silanes; crosslinking agents; and dyes. The composition can, of course, comprise several cosmetic ingredients appearing in the above list.

Depending on their nature and the purpose of the composition, the customary cosmetic ingredients can be present in normal amounts which can be easily determined by those skilled in the art and which can be, for each ingredient, between 0.01% and 80% by weight. Those skilled in the art will take care to choose the ingredients included in the composition and the amounts thereof so that they do not harm the properties of the compositions of the present invention.

The compositions in accordance with the invention may be packaged, for example, in a jar, in a tube, in a pump-dispenser bottle, in a foamer or in an aerosol device which is customary in the cosmetics industry.

The compositions according to the invention may, when they are intended to be packaged in an aerosol device, contain one or more propellant gases.

The propellant gas can then be chosen, for example, from volatile hydrocarbons, such as, in particular, C₁ to C₄ alkanes and preferably n-butane, propane, isobutane and mixtures thereof, chlorinated and/or fluorinated hydrocarbons, dimethyl ether and mixtures of these gases.

When it contains same, the composition comprises a propellant gas in a content ranging from 1% to 50% by weight and more preferentially from 1% to 10% by weight, relative to the total weight of said composition.

The compositions according to the invention may be, inter alia, in the form of liquids that are thickened to a greater or lesser extent, gels, creams, pastes or foams.

They are preferably in the form of foams generated by an aerosol or a foamer.

The cosmetic composition according to the invention may advantageously be used for the cosmetic treatment of the hair. In particular, it may be used for styling the hair, for example for shaping and/or fixing the hairstyle.

According to one particularly preferred embodiment, it is used for simultaneously styling and conditioning the hair.

The present invention also relates to a cosmetic treatment process for the hair, for example a hair care process, or a process for shaping and/or retaining the shape of the hairstyle, which consists in applying, to the hair, an effective amount of a composition as described above and then in optionally carrying out rinsing after an optional leave-on time.

Preferably, the composition according to the invention is not rinsed off.

The invention also relates to a cosmetic set for shaping keratin fibres, capable of forming a volume-expanded composition, comprising:
a composition such as that which has just been described; and
a volume-expanded composition dispenser for delivering said composition in the form of a volume-expanded composition.

The dispenser may be a bottle of foamer type or else an aerosol device, the composition then comprising, in the latter case, at least one propellent gas.

The dispensers of foamer type comprise a container for containing the composition and a dispensing head for delivering the composition. The foam is formed by forcing the composition to pass through a material comprising a porous substance such as a sintered material, a filtering grid made of plastic or of metal, or similar structures. The container comprises either a squeezable wall or a pump and a dip tube for transferring the composition from the container into the head in order to deliver the product.

The examples that follow are given as illustrations of the present invention. In these examples, all the amounts are indicated as weight percentages of active material (AM) relative to the total weight of the composition.

Foams in a Foamer

| | | Composi- | Composi- |
|---|---|---|---|
| INCI Name | Trade Names | tion 1 | tion 2 |
| VINYLAMINE/ | LUVIQUAT 9030 | 0.26 | 0.13 |
| VINYLFORMAMIDE | (BASF) (13% AM) | | |
| COPOLYMER | | | |
| VP/VA | LUVISKOL VA 64 W | — | 4 |
| COPOLYMER | (BASF) (50% AM) | | |
| PVP | PVP K30 L (ISP) | 2 | — |
| CETRIMONIUM | DEHYQUART A OR | — | 0.2 |
| CHLORIDE | (BASF) (25% AM) | | |
| HYDROXYETHYL | CHIMEXANE CL | 0.3 | — |
| OLEYL | (CHIMEX) | | |
| DIMONIUM | (30% AM) | | |
| CHLORIDE | | | |
| HYDROXYETHYL- | NATROSOL 250 | 0.05 | — |
| CELLULOSE | HHR PC | | |
| | (ASHLAND) | | |
| PROPYLENE | PROPYLENE | 2 | — |
| GLYCOL | GLYCOL USP/EP | | |
| | (DOW CHEMICAL) | | |
| PEG-40 | EUMULGIN HRE | 0.8 | 0.7 |
| HYDROGENATED | 40 (BASF) | | |
| CASTOR OIL | | | |
| FRAGRANCE | | 0.3 | 0.2 |
| PRESERVATIVES | | 0.7 | 0.8 |
| HYDROCHLORIC | SQ HYDROCHLORIC | — | 0.0000335 |
| ACID | ACID (QUALIGENS | | |
| | FINE CHEMICALS) | | |
| | (33.5% AM) | | |
| LACTIC | PURAC HS88 | 0.01 | — |
| ACID | (PURAC) | | |
| WATER | | qs 100 g | qs 100 g |

Pressurized foams

| | | Composi- | Composi- |
|---|---|---|---|
| INCI Name | Trade Names | tion 3 | tion 4 |
| VINYLAMINE/ | LUVIQUAT 9030 | 0.39 | 0.325 |
| VINYLFORMAMIDE | (BASF) (13% AM) | | |
| COPOLYMER | | | |
| VP/VA | LUVISKOL VA 64 W | 0.5 | — |
| COPOLYMER | (BASF) (50% AM) | | |
| PVP | PVP K30 L (ISP) | — | 2 |
| CETRIMONIUM | DEHYQUART A OR | 0.125 | 0.175 |
| CHLORIDE | (BASF) (25% AM) | | |
| QUATERNIUM-87 | VARISOFT W 575 | 0.375 | — |
| | PG N (EVONIK | | |
| | GOLDSCHMIDT) | | |
| | (75% AM) | | |
| HYDROXYETHYL- | NATROSOL 250 | — | 0.1 |
| CELLULOSE | HHR PC | | |
| | (ASHLAND) | | |
| PROPYLENE | PROPYLENE | 2.5 | — |
| GLYCOL | GLYCOL USP/EP | | |
| | (DOW CHEMICAL) | | |
| FRAGRANCE | | 0.3 | 0.1 |
| PEG-40 | EUMULGIN HRE | 0.6 | — |
| HYDROGENATED | 40 (BASF) | | |
| CASTOR OIL | | | |
| PRESERVATIVES | | 0.5 | — |
| ALCOHOL | ETHYL ALCOHOL | — | 8 |
| | SURFIN 99.9 | | |
| | DENATURED | | |
| | (FRANCE | | |
| | ALCOOLS) | | |
| HYDROCARBONS | PROPEL 45 | 4.5 | 4 |
| | (REPSOL) | | |
| DIMETHYL | DIMETHYL | 1.5 | — |
| ETHER | ETHER | | |
| | (SHELL) | | |
| WATER | | qs 100 g | qs 100 g |

Compositions 1 to 4 are prepared. On application to the hair, expanded foams are obtained which make it possible to obtain good styling and cosmetic properties with, in particular, long-lasting fixing over time and a pleasant feel.

1. Cosmetic composition comprising:
one or more vinylformamide/vinylformamine copolymers comprising:
from 10 to 95 mol % of units of the following formula A: 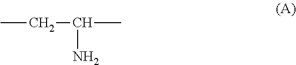
and from 90 to 5 mol % of units of the following formula B: 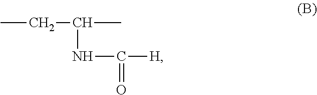
one or more non-ionic fixing polymers, and
one or more cationic surfactants.

2. Composition according to claim 1, characterized in that the vinylformamide/vinylformamine copolymer(s) comprise(s) from 10 to 60 mol % of units of formula A.

3. Composition according to the preceding claim, characterized in that the vinylformamide/vinylformamine copolymer(s) comprise(s) from 20 to 40 mol % of units of formula A.

4. Composition according to any one of the preceding claims, characterized in that the vinylformamide/vinylformamine copolymer(s) comprise(s) one or more additional monomer units, the latter representing less than 20 mol % of the copolymer.

5. Composition according to any one of claims 1 to 3, characterized in that the vinylformamide/vinylformamine copolymer(s) consist(s) solely of units of formula A and of units of formula B.

6. Composition according to any one of the preceding claims, characterized in that the vinylformamide/vinylformamine copolymer(s) is (are) present in proportions ranging from 0.01% to 20% by weight, more preferentially from 0.1% to 10% by weight and more particularly from 0.1% to 5% by weight, relative to the total weight of the composition.

7. Composition according to any one of the preceding claims, characterized in that the non-ionic fixing polymer(s) is (are) chosen from:
polyalkyloxazolines;
vinyl acetate homopolymers;
vinyl acetate copolymers;
homopolymers and copolymers of esters;
copolymers of acrylonitrile and of a non-ionic monomer;
styrene homopolymers;
styrene copolymers;
polyamides;
vinyllactam homopolymers;
vinyllactam copolymers;
non-hydrolysed polyvinylformamide polymers,
preferably from vinyllactam homopolymers and copolymers.

8. Cosmetic composition according to any one of the preceding claims, in which the non-ionic fixing polymer(s) is (are) present in a concentration ranging from 0.1% to 20% by weight, even better still from 0.2% to 10% by weight and even more preferentially from 0.5% to 5% by weight relative to the total weight of the composition.

9. Composition according to any one of the preceding claims, characterized in that the cationic surfactant(s) is (are) chosen from cetyltrimethylammonium, behenyltrimethylammonium and dipalmitoylethylhydroxyethylmethylammonium salts, quaternary ammonium salts of imidazoline, oleylhydroxyethyldimethylammonium salts, and mixtures thereof, and more particularly from behenyltrimethylammonium chloride, cetyltrimethylammonium chloride, methylalkylalkylamidoethylimidazolium methosulfate, oleylhydroxyethyldimethylammonium chloride, and mixtures thereof.

10. Composition according to any one of the preceding claims, characterized in that it comprises from 0.01% to 20% by weight, in particular from 0.1% to 10% by weight and better still from 0.15% to 5% by weight of cationic surfactant(s), relative to the total weight of the composition.

11. Composition according to any one of the preceding claims, characterized in that the cationic surfactant(s)/non-ionic fixing polymer(s) weight ratio ranges from 0.01 to 2 and even better still from 0.05 to 1.

12. Composition according to any one of the preceding claims, characterized in that it comprises water in a content ranging from 10% to 98%, preferably from 20% to 96%, better still from 50% to 96% by weight and even better still from 70% to 96% by weight relative to the total weight of the composition.

13. Composition according to any one of the preceding claims, characterized in that it is non-washing, i.e. it contains less than 5% by weight in total of anionic surfactants and of non-ionic surfactants.

14. Composition according to any one of the claims, characterized in that it comprises a propellant gas, the propellant gas being chosen from volatile hydrocarbons, such as, in particular, C₁ to C₄ alkanes and preferably n-butane, propane, isobutane and mixtures thereof, chlorinated and/or fluorinated hydrocarbons, dimethyl ether and mixtures of these gases, and preferably represents from 1% to 50% by weight and more preferentially from 1% to 10% by weight, relative to the total weight of said composition.

15. Cosmetic treatment process for the hair, characterized in that it consists in applying, to the hair, an effective amount of a composition according to any one of claims 1 to 14 and then in optionally carrying out rinsing after an optional leave-on time.

16. Cosmetic treatment process for the hair according to claim 15, characterized in that the application is carried out in the form of a foam.

17. Use of a composition according to any one of claims 1 to 14, for shaping and/or fixing the hairstyle.

18. Cosmetic set for shaping keratin fibres, capable of forming a volume-expanded composition, comprising:
a composition according to any one of claims 1 to 13; and
a volume-expanded composition dispenser for delivering said composition in the form of a volume-expanded composition.

19. Cosmetic set according to claim 18, in which the dispenser is an aerosol, the composition comprising at least one propellant gas.

20. Cosmetic set according to claim 18, in which the dispenser is a foamer.
